(19) United States
(12) Patent Application Publication
(10) Pub. No.: US 2004/0219068 A1
(43) Pub. Date: Nov. 4, 2004

(54) Intelligent microplates with remote data access

(76) Inventor: Peter Van Gerwen, Hever (BE)

Correspondence Address: William M Lee Jr, Barnes & Thornburg, P O Box 2786, Chicago, IL, 60690-2786, US

(21) Appl. No.: 10/482,563

(22) PCT Filed: Jun. 24, 2002
(86) PCT No.: PCT/EP02/07151

(30) Foreign Application Priority Data
Jun. 29, 2001 (EP) 01115918.3

(51) Int. Cl. G01N 33/00

(52) U.S. Cl. 422/82.12

(57) ABSTRACT

The present invention relates to a screening system comprising at least one array with a sensing means and a control unit comprising a first transceiving means and a data acquisition system which comprises a second transceiving means, wherein the first and second transceiving means interact through a wireless link.

### FIELD OF THE INVENTION

The present invention relates to a system and method for wireless interaction between the control unit and the data acquisition system of a screening device.

### BACKGROUND

In current screening set-ups, the sensitive element (B), which detects the signal generated by a sample in a recipient (A), is linked to a control unit (D), that collects the data and, depending on the type of sensitive element, drives and conditions an inducer signal. The collected data is then transferred electronically (by cabling, E) to a data acquisition system (F), whereby exact positioning towards the connector is required, necessitating actual contact. In most optical screening set-ups, also the precise alignment of the sensitive element (B) towards the recipient (A) is important to provide the optical path. The result is that these types of read-out systems usually require human intervention during multiple screening, which puts a constraint on high-throughput measurements, or measurements that need to be performed under specific conditions.

Another disadvantage of most current screening set-ups relates to the time dimension. In most optical, radioactive and other read-outs, a snap-shot recording of the situation is taken, thereby loosing all time-related information. This time dimension is of essence, for instance, to study agonist/antagonist mode of operations of ligand-receptor interactions. Time-continuous measurements can be made by leaving the microtiterplate in the read-out system. However, this causes a serious limitation on throughput. The plate ‘blocks’ the reader and other plates have to ‘wait’. Providing the system with a multitude of readers is a costly and unpractical alternative.

Additionally, the need for physical alignment of the different units in traditional read-out configurations puts a limitation on the environment wherein the measurements are performed. Measurements that require specific aberrant conditions (i.e. temperature or humidity) are either impossible or require specifically designed encasement for the plates.

As the integration of the both the sensitive element and the control unit together with the recipient in one microplate has never been conceived as a possibility, the physical alignment of the recipient (A) even possibly including the sensitive element (B) to the control unit (D) has always been required. Therefore, the physical connection to the data acquisition system has always been conceived as obvious, thereby imposing its limitation on the flexibility of the other units within the screening set-up. Thus, there has been little motivation to further integrate the different units into one element. Nevertheless, for many detection systems miniaturization and standardization of the sensors and/or control units have either been achieved or are readily conceivable. However, presently existing microtiterplates with integrated sensors, such as ISFETs, still require a physical alignment with the control unit (in this case the pH control unit) for data collection which is itself physically connected to the data acquisition system (F), so that the flexibility remains limited. According to the present invention however, the possibility to make the whole screening set-up completely flexible and independent from the data processor, makes complete integration of the different elements within one unit, preferably within the microtiterplate an attractive alternative.

Methods for wireless data transmission are known in the art. For instance, IrDA is an international organization that creates and promotes interoperable, low cost, infrared data interconnection standards. IrDA is a protocol designed to support transmission of data between two devices over short-range point-to-point infrared at speeds between 9.6 Kb/sec and 4 Mb/sec. IR transceiving still requires alignment, but it is far less stringent (usually detects at an angle of 30°).

The Bluetooth technology makes use of radio transmission in the 2.4 GHz ISM band. It is designed to operate in a noisy radio environment and to provide a fast, robust, and secure connection between devices. A full duplex data exchange rate of up to 1 Mb/s may be achieved. To date, the bluetooth technology has only found its adaptation in telecom and information technology.

The present invention provides for the integration of a wireless connection in a screening, a high-throughput screening or a high-requirement screening device. More specifically the present invention relates to microtiterplates with an integrated control unit (D) combined with a wireless link (E) to a data acquisition system (F).

The provision of a wireless connection for data transmission in a screening system provides complete flexibility to the screening process, making it worthwhile to integrate all other units so as to obtain an independent station, from which measurement data can be collected without limitations of time or physical positioning. The ability to ensure time-continuous measurements is a critical factor for the evaluation of many biological and biochemical interactions. Furthermore, the physical flexibility makes it possible to perform (high throughput) measurements of interactions or reactions under varying conditions without requiring specifically designed incubators.

### SUMMARY OF THE INVENTION

The present invention relates to a screening system or set-up comprising at least one array with a sensing means and a control unit comprising a first transceiving means and further comprising a data acquisition system which comprises a second transceiving means, wherein the first and second transceiving means interact through a wireless link.

According to a preferred embodiment of the invention, the wireless link is obtained through radio-frequency, preferably using the Bluetooth technology.

Alternatively, it can be envisaged that the wireless link is based on the detection of Infra-Red (IR) signals, such as described by IrDA.

The invention relates to a screening system that has a sensing means detecting a signal that can be converted to an electrical signal. The invention preferably relates to a screening system, which is a microcalorimetric device, comprising an array with a heat-sensing means. Preferably, the sensing means are thermopiles that directly generate an electrical signal. The sensing means is then connected to a control unit that is capable of transmitting data to the data acquisition system through a wireless link.

According a preferred embodiment of the invention, the screening system comprises an array, in which the sensitive element and the control unit, comprising the first transceiving means, are integrated into one microtiterplate-format device, and the screening system further comprises a data acquisition system comprising a second transceiving means, which interacts with the first transceiving means through a wireless link. According to another preferred embodiment said array additionally comprises a thermosetting means, which is also integrated into the microtiterplate.

According to another preferred embodiment of the invention, the array with the sensitive element and the control unit (and optionally the thermosetting means) of the screening system are monolithically integrated into one element.

According to a particular embodiment of the invention, microtiterplate devices comprising receiving means, sensing means, a control unit (and optionally thermosetting means) can be produced as single-use devices, from which data can be obtained directly by a data acquisition system through a wireless link with the control unit.

### DESCRIPTION OF FIGURES

The following detailed description, given by way of example, but not intended to limit the invention to specific embodiments described, may be understood in conjunction with the accompanying Figure, incorporated herein by reference, in which:

FIG. 1 represents the different elements of a standard screening set-up comprising a recipient (A), a sensitive element (B), a control unit (D) which interacts with the sensitive element (B) through a link (C), and a data acquisition system (F), which communicates with the control unit (D) through a link (E) which is mostly an electrical link. In a standard fluorescent screening set-up. The recipient (A) is a standard plastic microplate. The sensitive element (B) and the control unit (D) with their link (C) are integrated in a fluorescent reader. An alignment of the recipient (A) towards the sensitive element (C) is required: it provides an optical path. This fluorescent reader (B+C+D) is communicating with a data acquisition system (F) with electrical cables (E). In a pH screening set-up, the sensing element (B) is integrated in the recipient (A). The link (C) can be electrical or a combination of electrical and optical. The control unit (D) is a separate unit communicating with a data acquisition system (F) with electrical cables (E).

FIG. 2 represents different embodiments of the present invention;

FIG. 2a is a schematic representation of the preferred embodiment of the present invention. The plate includes three sub-parts. Sub-part (A+B) includes the recipients with integrated sensing elements. The sensing elements are preferentially thermopiles, but the set-up would also work for e.g. ISFETS, impedimetric sensors, SAW sensors, . . . etc. The link (C) is with ballbonds, pins, or any other electrical contact means. The sub-part (D+E) is an electronic PCB board including the control unit (D) including the transceiving means to provide the wireless link (E).

FIG. 2b represents an embodiment for an optical integrated system. The sub-part (A) includes only the recipients. The electronic PCB board (C+D+E) includes the control unit (D) with the transceiving means, and, it includes the sensing elements (B), optically aligned to the recipients in (A).

FIG. 2c represents an embodiment where everything is monolithically integrated.

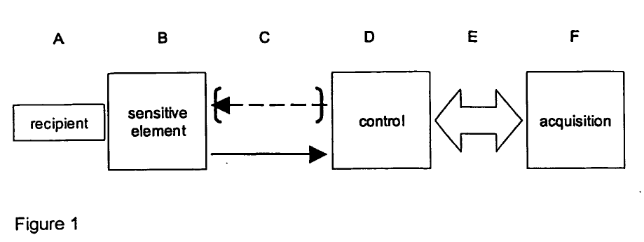

### DETAILED DESCRIPTION

The present invention relates to a system and method for screening or obtaining data by way of a screening device which is under the control of, and transmits data to a control unit through a wireless link.

The recipient (A), as used herein, refers to a recipient or receiving zone which can contain solvents with the biological, biochemical or chemical material in which the to be measured physical and/or chemical reactions and/or interactions are taking place.

An array as used herein refers to a series of at least two, but preferably a large number of recipients, which can hold a sample to be measured. Preferably, the array has the format of a standard microtiter plate, such as, but not limited to a 96-well, a 384-well, a 1536-well, a 3456-well, or a 6144-well microtiter plate. Alternatively, the design of the array can be customized for a specific type of sample or for integration into a specific robotic configuration which comprises, e.g. a dispensing device.

A signal as used herein refers to the physical and/or chemical effect of the physical and/or chemical reactions and/or interactions taking place within the recipient which can be detected by a sensing element as described hereafter.

The sensitive element or sensing means (B), as used herein, senses the reaction and/or interaction. The sensitive element can be sensing a variety of physical parameters including optical signals (fluorescence, luminescence, calorimetric information, . . . ), radio-active signals, thermal signals, electrical signals, . . . According to a preferred embodiment of the invention the sensing means comprises a heat detection means. Such heat detection means can be, but is not limited to a thermistor, diode, IR detection means, CCD camera, or a thermopile. According to a preferred embodiment of the present invention, the heat detection means is a differential heat detection means, such as a thermopile. Alternatively the sensing means comprises resistors. The heat detection means of the invention is capable of measuring the temperature of the sample in the recipient of the device. In the case of a differential heat detection means, a difference in temperature between samples in two neighboring recipients is measured.

The control unit (D), as used herein, conditions the signal and/or drives the sensitive element, if necessary. Its function can be limited to supplying the power to the sensitive element, provide it with a driving signal (a current, a laser excitation, . . . ) and/or condition a signal (e.g. a bias voltage, set a temperature, . . . ). The control unit (D) also ‘reads’ the signal from the sensor element. Possibly it is including some filtering, amplification and processing of the signal. Preferably the control unit can also send instructions to, and receive information from other devices, such as manipulation devices, which may or may not be part of the array. Possibly it includes transceiving means to communicate with the data acquisition system (F).

As, according to the invention, the signal to be transmitted should be an electrical signal, the control unit can additionally contain a sensor which converts the signal of the sensing means into an electrical signal. Most sensors (such as a CCD camera, a thermopile) directly generate an electrical signal. This electrical signal can be transmitted directly. In case of a sensor which detects an optical signal, this can be translated into an electrical signal (for further transmission to the data acquisition unit) by an additional sensor. According to the present invention the sensing means is connected to a transceiver capable of transmitting the data collected by the sensing means to a control unit through a wireless link.

A data acquisition system as used herein refers to a data processor, which is at least capable of processing data obtained from the sensing means.

In the screening set-up according to the present invention, the recipient (A), the sensing element (B), the link (C), and the control unit (D) are all integrated in the plate. The control unit (D) includes a transceiving means to realize a wireless link (E) with the data acquisition system (F).

The wireless link (E), as used herein, refers to the possibility to exchange data and information without an actual physical, electrical contact between the array or the sensing means therein, and the control unit. Preferably this is achieved by the transmission of data through radio-frequency. Most preferably, the wireless system used is the so-called “Bluetooth system”. By implementing this system, a transceiving means within the control unit (D) that is connected to the sensing means (B) in the array, can interact with a transceiving means connected to the data acquisition system (F). Thus, the transceiving means as used herein are capable of exchanging information without being electrically interconnected. Alternatively the wireless connection (E) can occur by sending data through infra-red wavelengths. The use of this system for a number of applications is described as IrDA.

The screening set-up of the invention can optionally further comprise a thermosetting means. A thermosetting means, as used herein, refers to a calibration means for thermosetting the recipients or receiving zones at a specific temperature. This calibration means can be a tuneable resistor or thermopile or a temperature sensitive means such as a resistor or diode or a microprocessor capable of driving a power generator in such a way that a predetermined temperature is obtained.

It is understood that the screening set-up of the present invention can further comprise, either integrated therein or be adapted to function within, manual or robotic manipulation devices, such as dispensers (for applying and removing fluids and samples) as well as other components which can be used in the manipulation of the samples or the generation of a suitable environment for the measurements required (e.g. gas flow, etc).

### PREFERRED EMBODIMENT OF THE INVENTION

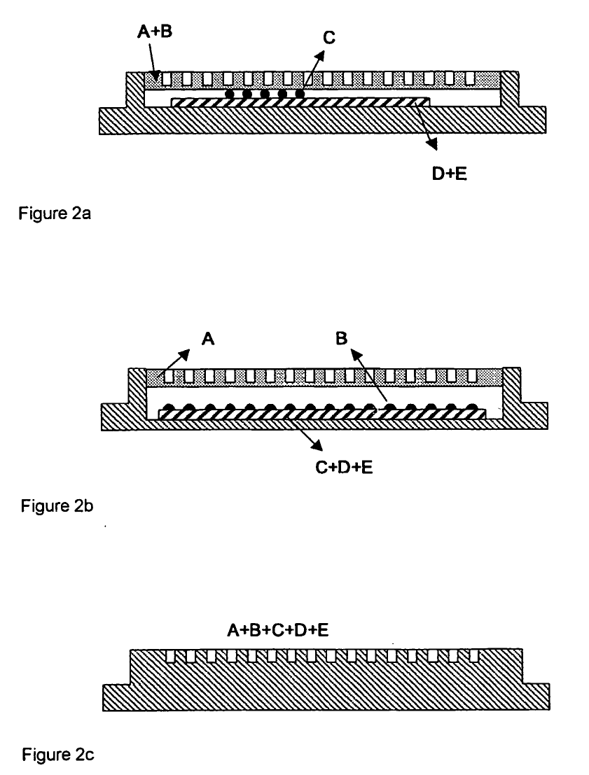

According to a preferred embodiment of the invention, the screening device is a microcalorimetric screening device and the sensing means a heat detection means. The array consists of a silicium chip which comprises a series of recipients which are in connection with the thermosensing means (as illustrated in FIG. 2a). The sensing means are electrically connected to a control unit comprising a Bluetooth transceiver, which transmits the data in a wireless way to a data acquisition system located nearby.

1. A screening system comprising
at least one array with sensing means
a control unit which reads the data of the sensing means, and, in the case of a non-electrical sensed signal, a means to translate the signal sensed by the sensing means into an electrical signal;
said control unit which, conditions the signal and/or drives the sensitive element;
said control unit comprising a first transceiving means; and
a data acquisition means comprising a second transceiving means
wherein said first and second transceiving means interact through a wireless link.

2. The screening system of claim 1, wherein said wireless link is based on data transmission through radio frequencies.

3. The screening system of claim 1, wherein said wireless link uses Bluetooth technology.

4. The screening system of claim 1, wherein said wireless link is based on data transmission at infra-red wavelengths.

5. The screening system of claim 4, wherein said wireless link makes use of the IrDA technology.

6. The screening system of claim 1, wherein the sensing means is a microcalorimetric sensing means.

7. The screening system of claim 1, wherein said at least one array also comprises a thermosetting means.

8. The screening system of claim 1, wherein said at least one array, said sensing means and said control unit are monolithically integrated.

9. An array comprising a sensing means and a control unit which reads the data of the sensing means, and, in the case of a non-electrical sensed signal, a means to translate the signal sensed by the sensing means into an electrical signal, whereby said control unit optionally conditions the signal and/or drives the sensitive element and said control unit comprises a first tranceiving means for interaction with a data acquisition means comprising a second transceiving means through a wireless link.

10. The array of claim 9, which has the format of a standard microtiter plate.
